(19) United States
(12) Patent Application Publication
(10) Pub. No.: US 2008/0082139 A1
(43) Pub. Date: Apr. 3, 2008

(54) Inhalation therapy using audiovisual stimuli

(76) Inventor: Mike John Means, Port Orchard, WA (US)

Correspondence Address: MICHAEL RIES, 318 PARKER PLACE, OSWEGO, IL, 60543, US

(21) Appl. No.: 11/542,286

(22) Filed: Oct. 2, 2006

(51) Int. Cl. A61N 1/00

(52) U.S. Cl. 607/54

(57) ABSTRACT

A method of improvement of inhalation therapy including presenting audiovisual stimuli to a patient, where content of the audiovisual stimuli promotes a physiological response, such as an involuntary breathing response, such as a yawn. Also, an apparatus for improvement of inhalation therapy including an audiovisual stimuli presentation system, where the audiovisual stimuli presentation system presents audiovisual information to a patient, and where content of the audiovisual stimuli presentation system promotes a physiological response, such as an involuntary breathing response, such as a yawn. The method and apparatus may be particularly suited for a patient that is unable or unwilling to communicate with a respiratory therapist, such as pediatric patients or patients with a neurological disorder.

### TECHNICAL FIELD & BACKGROUND

The present invention generally relates to the fields of inhalation therapy and incentive spirometry. More specifically, the present invention relates to a method of an apparatus for displaying images to augment spirometry, particularly for pediatric patients or patients who are unable or unwilling to communicate with a respiratory therapist.

There is a constant search in the inhalation therapy industry to find new ways to improve breathing function for patients undergoing inhalation therapy. Incentive spirometry, which is also known as sustained maximal inspiration, has been employed to improve breathing function. A variety of devices, known as incentive spirometers, have been developed, which are designed to provide positive feedback when certain characteristics of breathing are achieved, for example, the flow rate and duration of a breath. Typically, in inhalation therapy, a respiratory therapist works with a patient in order to achieve breathing at a desired rate and duration of breath in order to improve breathing function. Also, a spirometer and a clock might be used to show a patient what flow rate and duration are desired. However, some patients, such as pediatric patients including children less than the age of three, particularly infants, are not capable of understanding the instructions of a respiratory therapist, are not able to obtain feedback from measurement devices such as a spirometer or clock, or may not understand the purpose of incentive spirometry. Also, in some cases, patients may be unwilling to participate in activities with a respiratory therapist. Many types of respiratory therapy, including incentive spirometry, require the repetition of certain types of breathing over an extended period of time. Some patients, who might be able to understand the respiratory therapist, may refuse to follow the directions of the respiratory therapist. This may also be the case for adult patients experiencing neurological disorders, particularly for elderly patients experiencing, for example, dementia. As a result, respiratory therapists have difficulty achieving a desired response from the above-described types of patients.

The present invention delivers improved breathing function for patients undergoing inhalation therapy, particularly for patients unable to understand a respiratory therapist, such as infants, or adult and elderly patients with neurological disorders.

### DETAILED DESCRIPTION OF ILLUSTRATIVE EMBODIMENTS

Various aspects of the embodiments will be described using terms commonly employed by those skilled in the art to convey the substance of their work to others skilled in the art. However, it will be apparent to those skilled in the art that the present invention may be practiced with only some of the described aspects. For purposes of explanation, specific materials and configurations are set forth in order to provide a thorough understanding of the embodiments. However, it will be apparent to one skilled in the art that the present invention may be practiced without the specific details. In other instances, well-known features are omitted or simplified in order not to obscure the embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation. The phrase in one embodiment is used repeatedly. The phrase generally does not refer to the same embodiment, however, it may. The terms comprising, having and including are synonymous, unless the context dictates otherwise.

As noted above, incentive spirometry is often used in respiratory therapy as a means for improving breathing function and some patients are either incapable of or unwilling to work with a respiratory therapist. Specifically, some patients are unwilling or unable to voluntarily breathe in a prescribed manner, for a certain length of time at a specified flow rate.

The present invention improves respiratory therapy when combined with particular types of stimuli that provoke an involuntary need to yawn. Most humans, including infants, patients with severe neurological disorders or a patient that is unwilling to work with a therapist, are susceptible to a physiological response of yawning when they see and/or hear someone else yawning. When a person yawns, they take a deep breath and fill their lungs with air. Taking a deep breath increases oxygenation to the brain and heart and is very similar if not identical to the type of breathing desired in incentive spirometry. The present invention promotes healing in respiratory therapy by stimulating the patient with stimuli that provokes the physiological yawning response.

For example, the present invention may take the form of a type of media, such as a DVD disc or VHS tape, that includes images of, for example, humans, animals and/or animated characters yawning. Many hospital rooms include entertainment systems such as VCR or DVD players or video game systems. A patient may be exposed to these images and would, voluntarily or involuntarily, feel the need to yawn. As a result, air flow is increased throughout the lungs. The present invention includes all types of media and includes all types of audiovisual stimuli that provoke a physiological urge to yawn.

Specifically, the present invention is directed to a method of improvement of inhalation therapy. The method may comprise presenting audiovisual stimuli to a patient, where content of the audiovisual stimuli promotes a physiological response, such as an involuntary breathing response, such as a yawn.

The method is particularly suited for use with an infant patient, adult patients experiencing neurological disorders, elderly patients experiencing, for example, dementia, or any other patient that is unable or unwilling to communicate with a respiratory therapist.

For example an infant patient may watch a television connected to a DVD player displaying an image of a yawning cartoon character. An infant patient may watch a television connected to a DVD player displaying an image of a human baby yawning. A child may watch a television connected to a video game system and playing a game including images of a yawning character. An elderly patient may watch a television connected to a DVD player displaying an image of a yawning character.

The content may comprise audiovisual depictions of humans or animals yawning. The audiovisual depictions may comprise recordings of performances by live actors, live animals and/or animated characters.

The audiovisual stimuli may be stored on a DVD disc or VHS tape or a computer system. The computer system may be a video game system, and the audiovisual stimuli may be made to be part of a game that is played on the video game system. The game may be provided in an age-appropriate format.

The present invention is further directed to an apparatus for improvement of inhalation therapy. The apparatus may comprise an audiovisual stimuli presentation system, where the audiovisual stimuli presentation system presents audiovisual information to a patient, where content of the audiovisual stimuli presentation system promotes a physiological response, where the physiological response is an involuntary breathing response, and where the physiological response is a yawn.

As with the method described above, the apparatus may be suited for a patient that is unable or unwilling to communicate with a respiratory therapist, such as an infant.

As with the method described above, the content used with the apparatus may comprise audiovisual depictions of humans or animals yawning. The audiovisual depictions may comprise recordings of performances by live actors, live animals and/or animated characters. The audiovisual stimuli presentation system comprises a DVD disc or VHS tape, and the audiovisual information may be stored on the DVD disc or VHS tape or a computer system, such as a video game system.

While the present invention has been related in terms of the foregoing embodiments, those skilled in the art will recognize that the invention is not limited to the embodiments depicted. The present invention can be practiced with modification and alteration within the spirit and scope of the appended claims. Thus, the description is to be regarded as illustrative instead of restrictive on the present invention.

What is claimed is:

1. A method of improvement of inhalation therapy, the method comprising:
presenting audiovisual stimuli to a patient; and
promoting a physiological response from content of the audiovisual stimuli.

2. The method of claim 1, wherein the physiological response is an involuntary breathing response.

3. The method of claim 1, wherein the physiological response is a yawn.

4. The method of claim 1, wherein the patient is unable or unwilling to communicate with a respiratory therapist.

5. The method of claim 1, wherein the patient is an infant.

6. The method of claim 1, wherein the content comprises audiovisual depictions of a selected one of humans and animals yawning.

7. The method of claim 6, wherein the audiovisual depictions comprise recordings of performances by a selected one of live actors, live-animals and animation.

8. The method of claim 1, wherein the audiovisual stimuli are stored on a a selected one of DVD disc and VHS tape.

9. The method of claim 1, wherein the audiovisual stimuli are stored on a computer system.

10. The method of claim 9, wherein the computer system comprises a video game system,
wherein the audiovisual stimuli is part of a game played on the video game system.

11. An apparatus for improvement of inhalation therapy, the apparatus comprising:
an audiovisual stimuli presentation system;
the audiovisual stimuli presentation system presents audiovisual information to a patient; and
content of the audiovisual stimuli presentation system promotes a physiological response.

12. The apparatus of claim 11, wherein the physiological response is an involuntary breathing response.

13. The apparatus of claim 11, wherein the physiological response is a yawn.

14. The apparatus of claim 11, wherein the patient is a selected one of unable and unwilling to communicate with a respiratory therapist.

15. The apparatus of claim 11, wherein the patient is an infant.

16. The apparatus of claim 11, wherein the content comprises audiovisual depictions of a selected one of humans and animals yawning.

17. The apparatus of claim 16, wherein the audiovisual depictions comprise recordings of performances of a selected one of live actors, live animals and animation.

18. The apparatus of claim 11, wherein the audiovisual stimuli presentation system comprises a selected one of DVD disc and VHS tape,
wherein the audiovisual information is stored on the DVD disc or VHS tape.

19. The apparatus of claim 11, wherein the audiovisual stimuli presentation system comprises a computer system,
wherein the audiovisual information is stored on the computer system.

20. The apparatus of claim 19, wherein the computer system comprises a video game system,
wherein the audiovisual stimuli is part of a game played on the video game system.
